(19) United States
(12) Patent Application Publication
(10) Pub. No.: US 2006/0240080 A1
(43) Pub. Date: Oct. 26, 2006

(54) Alginate sponge and preparation method thereof

(76) Inventors: Seung-Man Han, Seoul (KR); Ik-Soo Kim, Gyeonggi-do (KR); Nam-Keun Han, Seoul (KR)

Correspondence Address: GIFFORD, KRASS, GROH, SPRINKLE & CITKOWSKI, P.C, PO BOX 7021, TROY, MI, 48007-7021, US

(21) Appl. No.: 10/549,002

(22) PCT Filed: Mar. 17, 2004
(86) PCT No.: PCT/KR04/00570

§ 371 Date: Nov. 3, 2005

(30) Foreign Application Priority Data
Mar. 18, 2003 (KR) 10-2003-0016849

(51) Int. Cl. A61K 38/20; A61K 38/19; A61K 38/18; A61K 9/70

(52) U.S. Cl. 424/443; 424/85.1; 424/85.2; 514/2

(57) ABSTRACT

The present invention relates to an alginate sponge and a preparation method thereof, more particularly to an alginate sponge having significantly improved flexibility, structural integrity, water-absorptivity, and processability, to be used for medical and tissue engineering purposes, and a simple preparation method thereof. The alginate sponge of the present invention has a maximum bend angle (flexibility) of at least 90°″ [symbol] , an apparent density (structural integrity) ranging from 0.006 to 0.1 glcm3, and a saline solution absorption ratio ranging from 150 to 700%.

### BACKGROUND OF THE INVENTION

(a) Field of the Invention

The present invention relates to an alginate sponge and a preparation method thereof, and more particularly to an alginate sponge having significantly improved flexibility, structural integrity, water-absorptivity, and processability, to be used for medical and tissue engineering purposes, and a simple preparation method thereof.

(b) Description of the Related Art

Alginic acid is a polysaccharide present in oceanic plants, corresponding to cellulose of land plants. It is a linear-chain copolymer comprising α-(1→4)-L-guluronic acid and β-(1→4)-D-mannuronic acid.

In general, alginic acid is the main constituent present between cell membranes of phaeophytae, which are the most productive oceanic plants. Commercially, it is obtained from laminaria, giant kelp, etc. It is widely used in the form of water-soluble sodium alginate. Water-soluble alginate derivatives are widely used as thickening agents, stabilizers, emulsifiers, and microcapsule materials in the fields of food, medicine, and fiber engineering because of their viscosity, biodegradability, non-toxicity, and easy gelation due to multivalent metal ions (e.g., Ca²⁺).

Additionally, there are many alginate products such as wound dressings and hemostatics utilizing the biodegradability, moisture absorptivity, hemostaticity, and biocompatibility of alginates.

As for alginate wound dressings, calcium alginate non-woven fiber made by spinning a sodium alginate solution in a coagulation bath of calcium chloride solution is the most popular. Examples are Kaltostat (Convatec, US), Sorbsan (Bertek, England), Nu-DERM (Johnson & Johnson, US), and Tegagen (3M, US). However, the non-woven type alginate wound dressings leave fiber debris when detached from the wound and are difficult to make into a variety of shapes. Accordingly, they are not suitable for tissue engineering.

To solve these problems, U.S. Pat. No. 3,653,383, U.S. Pat. No. 5,718,916, and U.S. Pat. No. 4,948,575 disclose methods of making an alginate sponge by adding a cross-linking agent to an alginate solution to form an alginate cross-linked gel, and then forming, freezing, and lyophilizing the same. However, the resultant alginate sponge offers bad tactility due to its rough surface. Also, it must be thicker than other sponges because its structure is not elaborate, or it has to be replaced frequently. Moreover, it has poor flexibility and poor adhesivity to a wound site, is brittle, has low fluidity, and thus has poor processability, so it is difficult to use as a wound dressing.

Accordingly, research on alginate sponges having superior flexibility, structural integrity, water-absorptivity, and processability to be used in the fields of medicine and tissue engineering are highly required.

### SUMMARY OF THE INVENTION

Thus, it is an object of the present invention to provide an alginate sponge having superior flexibility, structural integrity, water-absorptivity, and processability to be used in the fields of medicine and tissue engineering.

It is another object of the present invention to provide a method of preparing an alginate sponge having significantly improved flexibility, structural integrity, water-absorptivity, and processability to be used in the fields of medicine and tissue engineering by a simple process.

To attain the objects, the present invention provides an alginate sponge having a maximum bend angle (flexibility) of at least 90°, an apparent density (structural integrity) ranging from 0.006 to 0.1 g/cm³, and a saline solution absorption ratio ranging from 150 to 700%.

The present invention also provides a method of preparing an alginate sponge, comprising the steps of:

a) forming an alginate solution and freezing and lyophilizing it to prepare an alginate sponge medium; and

b) immersing said alginate sponge medium of step a) in a cross-linking agent solution and washing and drying the same to prepare an alginate sponge.

### BRIEF DESCRIPTION OF THE DRAWINGS

FIG. 1 is a cross-sectional optical micrograph of the alginate sponge prepared according to a preferred embodiment of the present invention, which shows the pore distribution.

FIG. 2 is another cross-sectional optical micrograph of the alginate sponge prepared according to a preferred embodiment of the present invention, which shows the pore distribution.

FIG. 3 is another cross-sectional optical micrograph of the alginate sponge prepared according to a preferred embodiment of the present invention, which shows the pore distribution.

FIG. 4 is a cross-sectional optical micrograph of the alginate sponge prepared according to the conventional method, which shows the pore distribution.

### DETAILED DESCRIPTION OF THE PREFERRED EMBODIMENTS

Hereinafter, the present invention is described in more detail.

The present inventors worked to develop an alginate sponge having superior flexibility, structural integrity, water-absorptivity and processability to be used in the filed of medicine and tissue engineering. In doing so, they found that an alginate sponge prepared by forming an alginate solution and freezing and lyophilizing it to prepare an alginate sponge medium, immersing said alginate sponge medium in a cross-linking agent solution, and then washing and drying it, has sufficient physical properties to be used in the fields of medicine and tissue engineering. That is, it has a maximum bend angle (flexibility) of at least 90°, an apparent density (structural integrity) ranging from 0.006 to 0.1 g/cm³, a saline solution absorption ratio ranging from 150 to 700%, and significantly improved flexibility, structural integrity, water-absorptivity, and processability.

The present invention is characterized by an alginate sponge having a maximum bend angle (flexibility) of at least 90°, an apparent density (structural integrity) ranging from 0.006 to 0.1 g/cm³, and a saline solution absorption ratio ranging from 150 to 700%.

If the maximum bend angle is below 90°, the alginate sponge is insufficiently flexible, so that it cannot be effectively applied on an indented wound site.

Also, if the apparent density is below 0.006 g/cm³, the alginate sponge becomes brittle due to its low structural integrity. Otherwise, if it exceeds 0.1 g/cm³, the surface of the alginate sponge becomes rough and the flexibility and saline solution absorption ratio decrease.

If the alginate sponge has a maximum bend angle and apparent density satisfying the above conditions, its saline solution absorption ratio ranges from 150 to 700%. Then, it can be effectively attached and applied on a wound site, and therefore it becomes a good wound dressing.

The present invention is also characterized by a method of preparing an alginate sponge comprising the steps of: forming an alginate solution and freezing and lyophilizing it to prepare an alginate sponge medium; and immersing said alginate sponge medium in a cross-linking agent solution and washing and drying it.

The method of preparing an alginate sponge according to the present invention is described in more detail.

a) Preparation of Alginate Sponge Medium

In this step, an alginate solution is formed, frozen, and lyophilized to prepare an alginate sponge medium.

Said alginate solution is prepared by dissolving alginate in pure water.

For the alginate, it is preferable to use alginic acid or alkali metal alginate, particularly sodium alginate, potassium alginate, or ammonium alginate, which are highly soluble in water.

Preferably, said alginate solution has an alginate content ranging from 0.5 to 30 wt %. If the alginate content is below 0.5 wt %, the low productivity increases production cost. Otherwise, if it exceeds 30 wt %, the high viscosity makes it difficult to remove air bubbles in the solution and decreases fluidity, so that processability and production reliability worsen.

Said alginate solution may further comprise a group 1A alkali metal salt, polyethylene oxide, polyvinyl alcohol, carboxymethylcellulose, carboxylated styrene butadiene latex, polyvinylpyrrolidone, coconut oil, glycerin, or surfactant to improve mechanical properties.

Also, said alginate solution may further comprise such bioactive factors as fibronectin, vitronectin, acidic fibroblast growth factor FGF, basic FGF, KGF, VEGF, EGF, PDFG-M, PDGF-AB, PDGF-BB, TGF-α, TGF-β, IGF, TNF, GM-CSF, NGF, heparin-binding EGF, interferon, erythropoietin, 1L-1 (interleukin-1), IL-2, IL-6, IL-8, and tissue-activated peptide. Said bioactive factors may be comprised alone or in combination.

Said alginate solution may be formed in a mold or by coating, die- casting, or extrusion. Preferably, said alginate solution is formed after removing air bubbles. Such formed alginate solution is promptly cooled to −10° C. or lower, and then frozen and lyophilized to prepare an alginate sponge medium.

The resultant alginate sponge medium has a fluidity superior to that of the conventional cross-linked alginate gel hydrate. Therefore, the alginate sponge prepared therefrom can be processed precisely and pores on the surface and inside of the sponge are fine and uniform, which improves flexibility.

b) Preparation of Alginate Sponge

In this step, the alginate sponge medium prepared in step a) is immersed in a cross-linking agent solution for cross-linking, and residual cross-linking agent is washed and dried to prepare an alginate sponge.

For the cross-linking agent comprised in the cross-linking agent solution, a divalent metal salt or organic cross-linking agent capable of covalent bonding can be used. To be specific, calcium chloride or zinc chloride may be used for the divalent metal salt, and glutaraldehyde, dicyclohexylcarbodiimide, or hexamethylene diisocyanate may be used for the organic cross-linking agent capable of covalent bonding.

The content of the cross-linking agent comprised in the cross-linking agent solution is not particularly limited. It can be varied depending on the requirement and apparent properties of the final alginate sponge product.

Also, said cross-linking agent solution may further comprise bioactive factors mentioned in step a), water-soluble chitosan, hyaluronic acid, pectin, or gelatin to offer anti-bacterial or skin regeneration effects.

After the alginate sponge medium is immersed in the cross-linking agent solution for cross-linking, it is washed to remove residual cross-linking agent and dried to prepare an alginate sponge having superior processability, flexibility, and uniform and integral pores.

Preferably, the resultant alginate sponge has a maximum bend angle (flexibility) of at least 90°, an apparent density (structural integrity) ranging from 0.006 to 0.1 g/cm³, and a saline solution absorption ratio ranging from 150 to 700%.

The alginate sponge of the present invention has such superior physical properties as flexibility, structural integrity, water-absorptivity, and processability in order to be used in the fields of medicine and tissue engineering. According to the present invention, the alginate sponge can be prepared by a simple process.

Hereinafter, the present invention is described in more detail through Examples. However, the following Examples are only for the understanding of the present invention and the present invention is not limited by the following Examples.

### EXAMPLES

### Example 1

Sodium alginate having medium viscosity (SIGMA) obtained from Macrocystis pyrifera (kelp) was dissolved in pure water to 1 wt % to prepare an alginate solution. Air bubbles were completely removed from the solution under reduced pressure. Then, the alginate solution was put in a plastic petri dish (diameter=100 mm), frozen at −40° C. and lyophilized to prepare a sodium alginate sponge medium.

The resultant sodium alginate sponge medium was immersed in a 0.2 M calcium chloride solution for 30 minutes for cross-linking. Then, it was washed with deionized water several times to remove residual cross-linking agent. Then, it was frozen at −40° C. again and lyophilized to prepare a water-insoluble cross-linked calcium alginate sponge.

### Example 2

A water-insoluble cross-linked calcium alginate sponge was prepared in the same manner of Example 1, except for using ammonium alginate (CarboMer, US) instead of sodium alginate.

### Example 3

A water-insoluble cross-linked calcium alginate sponge was prepared in the same manner of Example 1, except for using potassium alginate (Kimica, Japan) instead of sodium alginate.

### Comparative Example 1

15 mL of 0.2M calcium chloride solution was slowly added dropwise to 300 g of the sodium alginate solution (sodium alginate content=1 wt %) prepared in Example 1 while stirring to prepare a cross-linked alginate gel. The resultant cross-linked alginate gel was put in a 100 mm plastic petri dish and frozen at −40° C. and lyophilized to prepare a water-insoluble cross-linked calcium alginate sponge.

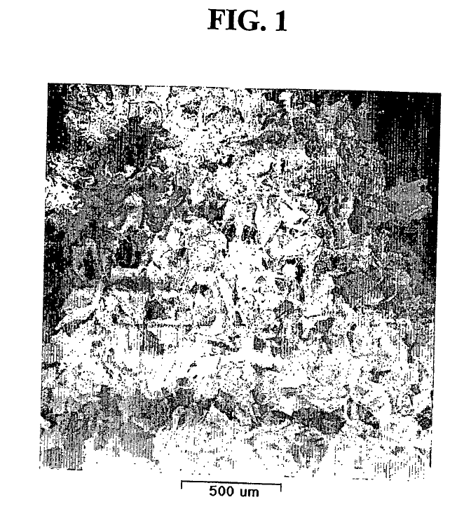

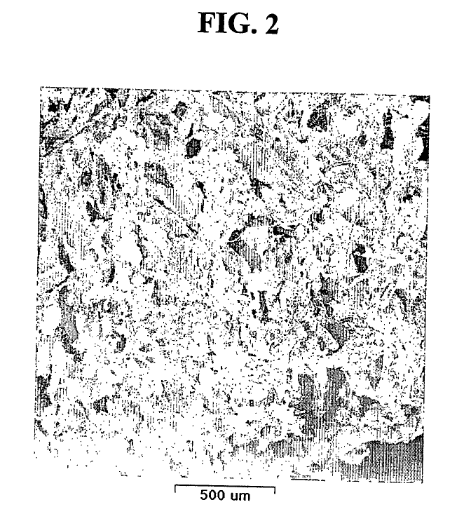

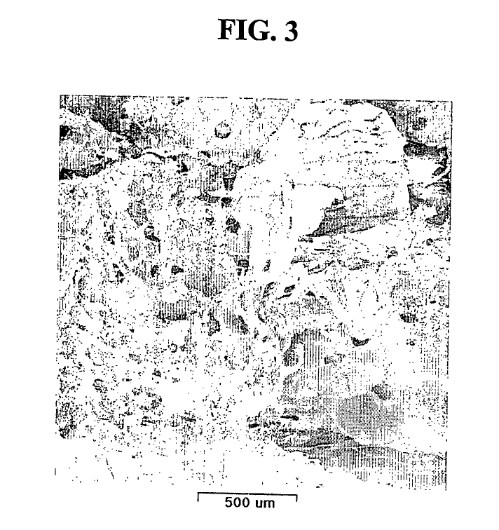

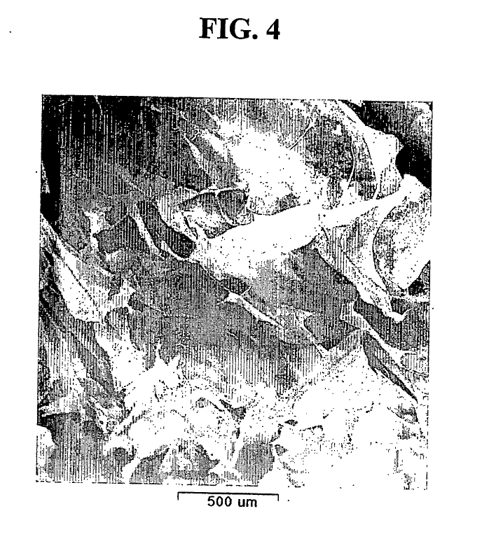

Cross-sections of alginate sponges prepared in Examples 1 to 3 and Comparative Example 1 were observed with an optical microscope. The results are shown in FIGS. 1 to 4. Flexibility, average pore size, and water absorption ratio were measured as follows. The results are shown in Table 1 below.

a) Flexibility (°)—The maximum bend angle without sponge breaking was determined.

b) Average pore size (g/cm³)—Apparent density was measured and compared.

c) Water absorption ratio (%)—Sponge sample was dried in a desiccator at 60° C. for 24 hours and weighed (weight=A). It was immersed in a 0.9% sodium chloride solution at 25° C. for 48 hours. Then, it was centrifuged at 160 G for 5 minutes and weighed (weight=B). The water absorption ratio was determined by the following Equation 1:

$$Water absorption ratio ( \% ) = [ \frac{( B - A )}{A} ] \times 100 \qquad Equation 1$$

TABLE 1

| | | | | Comparative |
|---|---|---|---|---|
| Classification | Example 1 | Example 2 | Example 3 | Example 1 |
| Flexibility (°) | 150 | 140 | 145 | 70 |
| Average pore size | 0.0377 | 0.0341 | 0.0384 | 0.0046 |
| (g/cm³) | | | | |
| Water absorption | 306 | 268 | 289 | X |
| ratio (%) | | | | |

As seen in Table 1 and FIGS. 1 to 4, the alginate sponges prepared according to the present invention (Examples 1 to 3) had superior flexibility, average pore size (structural integrity), and water absorption ratio than the alginate sponge prepared according to the conventional method (Comparative Example 1). In addition, while the alginate sponges of the present invention (Examples 1 to 3) maintain the sponge structure and offer a high saline solution absorption ratio, the alginate sponge of Comparative Example 1 experienced structure breakage as the saline. solution absorption ratio increased.

Therefore, the present invention provides an alginate sponge having significantly improved physical properties such as flexibility, structural integrity, water-absorptivity, and processability to be used in the fields of medicine and tissue engineering, and which is prepared by a simple process.

While the present invention has been described in detail with reference to the preferred embodiments, those skilled in the art will appreciate that various modifications and substitutions can be made thereto without departing from the spirit and scope of the present invention as set forth in the appended claims.

What is claimed is:

1. An alginate sponge having a maximum bend angle (flexibility) of at least 90°, an apparent density (structural integrity) ranging from 0.006 to 0.1 g/cm³, and a saline solution absorption ratio ranging from 150 to 700%.

2. A method of preparing an alginate sponge comprising the steps of:
a) forming an alginate solution and freezing and lyophilizing the same to prepare an alginate sponge medium; and
b) immersing said alginate sponge medium in a cross-linking agent solution for cross-linking, and washing and drying the same to prepare an alginate sponge.

3. The method of preparing an alginate sponge of claim 2, wherein said alginate solution of step a) comprises 0.5 to 30 wt % of alginic acid or alkali metal alginate selected from the group consisting of sodium alginate, potassium alginate, and ammonium alginate.

4. The method of preparing an alginate sponge of claim 2, wherein said alginate solution of step a) further comprises one or more materials selected from the group consisting of alkali metal salt, polyethylene oxide, polyvinyl alcohol, carboxymethylcellulose, carboxylated styrene butadiene latex, polyvinylpyrrolidone, coconut oil, glycerin, and surfactant.

5. The method of preparing an alginate sponge of claim 2, wherein said alginate solution of step a) further comprises one or more bioactive factors selected from the group consisting of fibronectin, vitronectin, acidic fibroblast growth factor FGF, basic FGF, KGF, VEGF, EGF, PDFG-M, PDGF-AB, PDGF-BB, TGF-α, TGF-β, IGF, TNF, GM-CSF, NGF, heparin-binding EGF, interferon, erythropoietin, 1L-1 (interleukin-1), IL-2, IL-6, IL-8, and tissue-activated peptide.

6. The method of preparing an alginate sponge of claim 2, wherein said cross-linking agent comprised in said cross-linking agent solution of step b) is a divalent metal salt or an organic cross-linking agent capable of covalent bonding.

7. The method of preparing an alginate sponge of claim 2, wherein said cross-linking agent solution of step b) further comprises one or more materials selected from the group consisting of a bioactive factor, water-soluble chitosan, hyaluronic acid, pectin and gelatin.

8. The method of preparing an alginate sponge of claim 2, wherein said alginate sponge has a maximum bend angle (flexibility) of at least 90°, an apparent density (structural integrity) ranging from 0.006 to 0.1 g/cm³, and a saline solution absorption ratio ranging from 150 to 700%.
